(19) United States
(12) Patent Application Publication
(10) Pub. No.: US 2006/0211882 A1
(43) Pub. Date: Sep. 21, 2006

(54) Method for producing optically active carboxylic acid

(75) Inventors: Akira Amano, Kanagawa (JP); Daisuke Igarashi, Kanagawa (JP); Noboru Sayo, Kanagawa (JP)

Correspondence Address: EDWARDS & ANGELL, LLP, P.O. BOX 55874, BOSTON, MA, 02205, US

(73) Assignee: Takasago International Corporation, Tokyo (JP)

(21) Appl. No.: 10/550,564

(22) PCT Filed: Mar. 26, 2004
(86) PCT No.: PCT/JP04/04373

§ 371 Date: Sep. 26, 2005

(30) Foreign Application Priority Data
Mar. 28, 2003 (JP) 2003-089605

(51) Int. Cl. C07C 51/36

(52) U.S. Cl. 562/401; 562/606

(57) ABSTRACT

A method for producing a desired optically active carboxylic acid with a high optical purity, wherein a complex catalyst used can be recovered and reused as an aqueous solution. The method contains the step of subjecting an &agr;,&bgr; -unsaturated carboxylic acid in water or a mixed solvent of water and a water-insoluble organic solvent in the presence of a sulfonated BINAP—Ru complex represented by the formula [3]: [RuX(arene){(SO?3#191M)?2#191-BINAP}]X [3]wherein X represents a chlorine atom, a bromine atom or an iodine atom, arene represents a benzene or an alkyl-substituted benzene, M represents an alkaline metal atom, and BINAP represents 2,2′-bis(diphenylphosphine)-1,1′-binaphthyl to an asymmetric hydrogenation. The sulfonated BINAP—Ru complex can be recycled.

### BACKGROUND OF THE INVENTION

1. Field of the Invention

The present invention relates to a method for producing an optically active carboxylic acid useful as a pharmaceutical intermediate, a liquid crystal material, perfumes, etc.

2. Description of the Related Art

Generally, most of pharmaceutical intermediates are solid and it is difficult to separate a pharmaceutical intermediate from a catalyst by distillation. Separation of catalysts and products is one of unavoidable problems. Particularly, catalysts for use in homogeneous catalytic reactions are easily dissolved in organic phases, so that complicated procedures such as distillation and recrystallization are required to separate such catalysts and products. One solution of the problem is a method in which a reaction is carried out in a water-containing solvent using a water-soluble catalyst. In this method, the catalyst can be easily separated only by extraction because the product is dissolved in the organic phase and the catalyst is dissolved in the water phase. Water-soluble phosphine ligands have attracted attention as the water-soluble catalyst, and many reports have been made thereon.

Asymmetric hydrogenation of ketones and imines using a sulfonated BINAP are described in JP-A-5-170780. However, asymmetric hydrogenation of olefins are not described in the patent document, and reuse of the catalyst dissolved in water, which is used in the reaction once, is also not described.

An example of synthesizing anti-inflammatory analgesic drug naproxen has been reported in J. Catal., Vol. 148, Page 1, 1994. A ligand used in the synthesis is such that BINAP (2,2′-bis(diphenylphosphine)-1,1′-binaphthyl) is sulfonated to have sulfone groups at all the meta positions of 4 phenyl groups. The ligand is converted to a ruthenium complex and used for hydrogenating dehydronaproxen. Though the enantiomer excess of naproxen produced by the asymmetric hydrogenation in methanol is 96.1% ee, the enantiomer excess is considerably reduced to 77.6% ee in the case of the asymmetric hydrogenation in water/methanol.

Asymmetric hydrogenation of dehydronaproxen in water/ethyl acetate and recycle of the water phase are also described in J. Catal., Vol. 148, Page 1, 1994. However, the enantiomer excess of naproxen obtained by the asymmetric hydrogenation is 81.1% ee, and the enantiomer excess is insufficiently 82.7% ee in the case of recycling the water phase. Further, it takes 1.5 days to complete the asymmetric hydrogenation, whereby the synthesis method disadvantageously needs improvement of workability.

An example of asymmetric hydrogenation of tiglic acid is described in J. Mol. Cat., Vol. 159, Page 37, 2000. A ruthenium complex used in the asymmetric hydrogenation contains a ligand obtained by aminating carbon atoms at 5,5′-positions of BINAP and by introducing polyethylene glycol, etc. to make the BINAP water-soluble. The asymmetric hydrogenation is carried out in a two-phase system of ethyl acetate/water solvent, and as a result, the enantiomer excess of the product is insufficiently 83% ee. Experiments of recycling the ruthenium complex catalyst are not described in the reference.

As described above, though many reports have been made on asymmetric hydrogenation methods using water-soluble phosphine ligands in two-phase systems of water and organic phases, most of the methods are disadvantageous in enantiomer excess and catalytic activity to be impractical. Further, most of the methods are unsatisfactory in view of separation of products and catalysts, reuse of catalysts, etc. depending on intended reactions and substrates. The ligands and the transition metals contained in the optically active complex catalysts are extremely expensive, whereby it has been desired to develop a synthesis method capable of recycling the catalyst to most efficiently reduce production costs.

### SUMMARY OF THE INVENTION

An object of the present invention is to provide a method capable of producing a desired optically active carboxylic acid with a high optical purity, wherein a complex catalyst used can be recovered as an aqueous solution and the recovered complex catalyst solution can be recycled, in view of the above-described situation.

A first method of the present invention for producing an optically active carboxylic acid represented by the formula [2]: 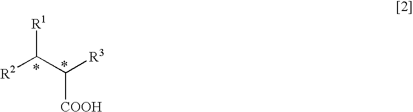
wherein R¹, R² and R³ independently represent a hydrogen atom, an alkyl group, an alkenyl group or an aryl group, the groups may have a substituent, R¹, R² and R³ is not a hydrogen atom simultaneously, R³ is a group other than a hydrogen atom when one of R¹ and R² is a hydrogen atom, R³ is a group other than a hydrogen atom and a methyl group when both of R¹ and R² are hydrogen atoms, and R¹ and R² are different groups other than a hydrogen atom when R³ is a hydrogen atom, and at least one of the two carbon atoms marked with * represents an asymmetric carbon atom, comprising the step of subjecting an α,β-unsaturated carboxylic acid represented by the formula [1]: 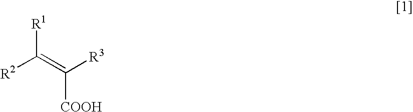
wherein R¹ to R³ have the same meanings as those in the formula [2], in the presence of a sulfonated BINAP—Ru complex represented by the formula [3]:
[RuX(arene){(SO₃M)₂-BINAP}]X [3]
wherein (SO₃M)₂-BINAP represents a tertiary phosphine represented by the general formula [4]: 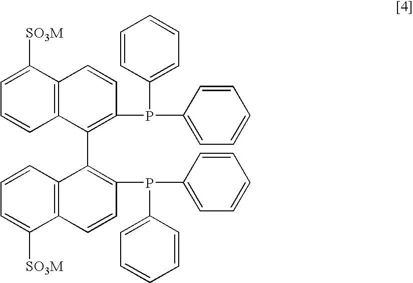
M represents an alkaline metal atom, X represents a chlorine atom, a bromine atom or an iodine atom, and arene represents a benzene or an alkyl-substituted benzene, in an aqueous solvent, to an asymmetric hydrogenation.

A second method of the invention for producing an optically active carboxylic acid represented by the formula [2]: 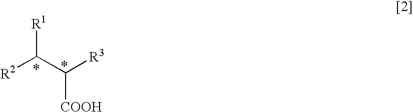
wherein R¹, R² and R³ independently represent a hydrogen atom, an alkyl group, an alkenyl group or an aryl group, the groups may have a substituent, R¹, R² and R³ is not a hydrogen atom simultaneously, R³ is a group other than a hydrogen atom when one of R¹ and R² is a hydrogen atom, R³ is a group other than a hydrogen atom and a methyl group when both of R¹ and R² are hydrogen atoms, and R¹ and R² are different groups other than a hydrogen atom when R³ is a hydrogen atom, and at least one of the two carbon atoms marked with * represents an asymmetric carbon atom, comprising the step of subjecting an α,β-unsaturated carboxylic acid represented by the formula [1]: 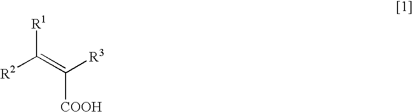
wherein R¹ to R³ have the same meanings as those described above, in the presence of a recovered sulfonated BINAP—Ru complex used in the first method in water or a mixed solvent of water and a water-insoluble organic solvent to an asymmetric hydrogenation.

Thus, as a result of intense research in view of the above object, the inventors have found that an optically active carboxylic acid with a high optical purity can be obtained by asymmetric hydrogenation of the α,β-unsaturated carboxylic acid using the sulfonated BINAP—Ru complex represented by the formula [3] in an aqueous solvent such as water or the mixed solvent of water and the water-insoluble organic solvent and that the complex catalyst can be recycled while maintaining high catalytic activity. The invention has been achieved by the findings.

### DESCRIPTION OF THE PREFERRED EMBODIMENTS

In the formulae [1] and [2], the alkyl group represented by R¹, R² or R³ may be a linear, branched or cyclic alkyl group having a carbon number of 1 to 20, preferably 1 to 15, more preferably 1 to 10. Specific examples of the alkyl groups include a methyl group, an ethyl group, a n-propyl group, a 2-propyl group, a n-butyl group, a 2-butyl group, an isobutyl group, a tert-butyl group, a n-pentyl group, a 2-pentyl group, a 2-methylbutyl group, a 3-methylbutyl group, a 2,2-dimethylpropyl group, a n-hexyl group, a 2-hexyl group, a 3-hexyl group, a 2-methylpentane-2-yl group, a 3-methylpentane-3-yl group, a 2-methylpentyl group, a 3-methylpentyl group, a 4-methylpentyl group, a 2-methylpentane-3-yl group, a heptyl group, an octyl group, a 2-ethylhexyl group, a nonyl group, a decyl group, a cyclopropyl group, a cyclobutyl group, a cyclopentyl group, a cyclohexyl group, etc.

The alkenyl group represented by R¹, R² or R³ may be such that 1 or more double bond is introduced to the above alkyl groups having 2 or more carbon atoms. Specific examples of the alkenyl groups include an ethenyl group, a 1-propenyl group, a 2-propenyl group, an isopropenyl group, a 1-butenyl group, a 2-butenyl group, a 1,3-butadienyl group, a 2-pentenyl group, a 2-hexenyl group, a heptenyl group, an octenyl group, a nonenyl group, a decenyl group, a cyclopropenyl group, a cyclopentenyl group, a cyclohexenyl group, etc.

The aryl group represented by R¹, R² or R³ may be an aryl group having 6 to 14 carbon atoms. Specific examples of the aryl groups include a phenyl group, a naphthyl group, an anthryl group, a biphenyl group, etc.

The substituent bonding to the alkyl, alkenyl or aryl group, i.e. substituent of a substituted alkyl group, a substituted alkenyl group or a substituted aryl group, may be any group that has no adverse affect on the asymmetric hydrogenation of the invention, and examples thereof include alkyl groups, alkoxy groups, aryl groups, halogen atoms, etc.

The meanings and specific examples of the alkyl groups and the aryl groups as the substituent may be the same as those described above.

The alkoxy group may be a linear, branched or cyclic group having 1 to 20, preferably 1 to 10, more preferably 1 to 6 carbon atoms. Specific examples of the alkoxy groups include a methoxy group, an ethoxy group, a n-propoxy group, a 2-propoxy group, a n-butoxy group, a 2-butoxy group, an isobutoxy group, a tert-butoxy group, a n-pentyloxy group, a 2-methylbutoxy group, a 3-methylbutoxy group, a 2,2-dimethylpropyloxy group, a n-hexyloxy group, a 2-methylpentyloxy group, a 3-methylpentyloxy group, a 4-methylpentyloxy group, a 5-methylpentyloxy group, a cyclohexyloxy group, etc.

Examples of the halogen atoms include a fluorine atom, a chlorine atom, a bromine atom, an iodine atom, etc.

In the formulae [1] and [2], R¹, R² and R³ represent the above atom or group respectively, and it should be noted that R¹, R² and R³ is not a hydrogen atom simultaneously based on the definition that at least one of the two carbon atoms marked with * in the formula [2] represents an asymmetric carbon atom. Further, R³ is a group other than a hydrogen atom when one of R¹ and R² is a hydrogen atom, R³ is a group other than a hydrogen atom and a methyl group when both of R¹ and R² are hydrogen atoms, and R¹ and R² are different groups other than a hydrogen atom when R³ is a hydrogen atom.

This is because, in the formula [2], the carbon atom bonding to R¹ and R² is not an asymmetric carbon atom in the case where R¹ and/or R² is a hydrogen atom, and the carbon atom bonding to R³ is not an asymmetric carbon atom in the case where R³ is a hydrogen atom or in the case where both of R¹ and R² are hydrogen atoms and R³ is a methyl group.

In the formula [3], arene represents a benzene or an alkyl-substituted benzene. Examples of preferred alkyl-substituted benzenes include p-cymene, hexamethylbenzene, 1,3,5-trimethylbenzene, etc.

In the formulae [3] and [4], the alkaline metal atom represented by M may be a sodium atom, a potassium atom, etc.

Specific examples of the α,β-unsaturated carboxylic acids represented by the formula [1], used as a starting material in the methods of the invention, include 2-methylbutenoic acid, 2-methyl-2-pentenoic acid, 2-methyl-2-hexenoic acid, 2-ethyl-2-hexenoic acid, 2-methyl-2-heptenoic acid, 2-methyl-2-octenoic acid, etc.

Specific examples of the sulfonated BINAP—Ru complexes represented by the formula [3] used in the methods of the invention include [RuI(p-cymene) {(SO₃Na)₂—BINAP}]I, [RuBr(p-cymene) {(SO₃Na)₂—BINAP}]Br, [RuCl(p-cymene) {(SO₃Na)₂—BINAP}]Cl, [RuI(C₆H₆){(SO₃Na)₂—BINAP}]I, [RuBr(C₆H₆) {(SO₃Na)₂—BINAP}]Br, [RuCl(C₆H₆){(SO₃Na)₂—BINAP}]Cl, etc.

The sulfonated BINAP—Ru complexes can be easily produced by the methods described in JP-A-5-170780.

Specific examples of the optically active carboxylic acids represented by the formula [2], obtainable by the methods of the invention, include (2R)-methylbutanoic acid, (2R)-methylpentanoic acid, (2R)-methylhexanoic acid, (2R)-ethylhexanoic acid, (2R)-methylheptanoic acid, (2R)-methyloctanoic acid, (2S)-methylbutanoic acid, (2S)-methylpentanoic acid, (2S)-methylhexanoic acid, (2S)-ethylhexanoic acid, (2S)-methylheptanoic acid, (2S)-methyloctanoic acid, etc.

In the methods of the invention, the mole ratio of the sulfonated BINAP—Ru complex represented by the formula [3] to the α,β-unsaturated carboxylic acid is appropriately selected generally from the range of 1×10⁻² to 3×10⁻⁴ mol/mol, preferably from the range of 1×10⁻³ to 2×10⁻⁴ mol/mol.

In the methods of the invention, the asymmetric hydrogenation is carried out in an aqueous solvent. The aqueous solvent is water or the two-phase mixed solvent of water and the water-insoluble organic solvent.

Specific examples of the water-insoluble organic solvents used in the methods of the invention include aliphatic hydrocarbons such as pentane, hexane heptane, octane, decane, and cyclohexane; halogenated hydrocarbons such as methylene chloride, 1,2-dichloroethane, chloroform, carbon tetrachloride, and 1,2-dichlorobenzene; ethers such as diethyl ether, diisopropyl ether, dimethoxyethane, ethylene glycol diethyl ether, tert-butyl methyl ether, and cyclopentyl methyl ether; esters such as methyl acetate, ethyl acetate, n-butyl acetate, and methyl propionate; etc. These solvents may be used alone or in appropriate combination of two or more solvents thereof.

The amount of the water-insoluble organic solvent is appropriately selected generally from the range of 1 to 10 parts by weight, preferably from the range of 2 to 5 parts by weight, per 1 part by weight of the α,β-unsaturated carboxylic acid.

Water used in the methods of the invention may be distilled water, purified water, ion-exchange water, etc. Water is preferably distilled and degassed.

The amount of water is appropriately selected generally from the range of 1 to 25 parts by weight, preferably from the range of 1 to 15 parts by weight, per 1 part by weight of the α,β-unsaturated carboxylic acid. The amount of water remarkably affects the asymmetric hydrogenation rate depending on the carbon number of the α,β-unsaturated carboxylic acid. The amount of water may be 1 to 2 parts by weight in the case of tiglic acid having 5 carbon atoms, and the amount is 10 parts or more by weight in the case of 2-ethylhexenoic acid having 8 carbon atoms.

In the asymmetric hydrogenation of the invention, the hydrogen pressure is desirably 0.1 MPa or more, and appropriately selected generally from the range of 0.5 to 10 MPa, preferably from the range of 1 to 5 MPa, from the viewpoint of economical efficiency, etc.

The reaction temperature in the methods of the invention is appropriately selected generally from the range of 30 to 100° C., preferably from the range of 40 to 90° C.

The reaction time depends on the conditions such as the reaction temperature, the amount of the sulfonated BINAP—Ru complex, the amount of water, and the hydrogen pressure. The reaction time is appropriately selected generally from the range of 1 to 20 hours, preferably from the range of 3 to 10 hours.

In the methods of the invention, an aqueous solution of the sulfonated BINAP—Ru complex used in the asymmetric hydrogenation can be recovered and reused.

Thus, the sulfonated BINAP—Ru complex can be recycled (reused) in the methods of the invention.

The sulfonated BINAP—Ru complex or the aqueous solution thereof may be recovered by a common operation from the reaction solution (reaction system).

Specifically, the aqueous solution of the sulfonated BINAP—Ru complex may be recovered by separating the water phase from the two-phase reaction solution after the asymmetric hydrogenation.

Further, the sulfonated BINAP—Ru complex can be easily recovered from the separated water phase by concentration, etc.

The recovered aqueous solution of the sulfonated BINAP—Ru complex (the water phase separated after the asymmetric hydrogenation) may be directly reused (recycled) without aftertreatments and purifications for the asymmetric hydrogenation of the α,β-unsaturated carboxylic acid.

The isolated or recovered sulfonated BINAP—Ru complex may be reused for the asymmetric hydrogenation of the α,β-unsaturated carboxylic acid or for other asymmetric hydrogenation after aftertreatment, purification, etc.

In the case where the recovered sulfonated BINAP—Ru complex, which may be the water phase containing the sulfonated BINAP—Ru complex recovered from the reaction solution (reaction system) or the sulfonated BINAP—Ru complex isolated from the water phase, is recycled for the asymmetric hydrogenation of the α,β-unsaturated carboxylic acid to produce the optically active carboxylic acid, the amount of the sulfonated BINAP—Ru complex may be appropriately controlled if necessary by adding further sulfonated BINAP—Ru complex, etc.

Thus-obtained optically active carboxylic acid is useful as pharmaceutical intermediates, liquid crystal materials, etc.

### EXAMPLES

The present invention will be described in more detail below with reference to Examples without intention of restricting the scope of the invention.

In Examples, physical properties are measured by the following apparatuses.

1) Chemical Purity

Gas chromatography (GLC): TC-WAX column.

2) Optical Purity

Carboxylic acids were converted to L-(−)-1-phenylethylamides to measure the optical purities.

Gas chromatography (GLC): Chiraldex G-PN column.

3) Optical Rotation

JASCO DIP-360 polarimeter.

4) Mass Spectrum

Shimadzu GC-MS-QP2010.

GLC column: TC-WAX.

### Example 1

### Synthesis of (2R)-methylbutanoic acid

10 g (0.1 mol) of tiglic acid (available from Tokyo Kasei Kogyo Co., Ltd.) and 8.7 mg (6.6×10⁻³ mmol) of [RuI(p-cymene){(R)—(SO₃Na)₂BINAP}]I were put in a 200 mL autoclave, and the atmosphere in the autoclave was replaced with nitrogen. 20 mL of methylene chloride, which was degassed and distilled while blocking air flow by nitrogen, and 10 mL of degassed distilled water were added to the mixture, and tiglic acid was reacted at 80° C. for 4 hours under the hydrogen pressure of 2.5 MPa. The temperature of the autoclave was lowered to the room temperature, hydrogen was discharged, and nitrogen was flowed in the autoclave for approximately 30 minutes to remove the remaining hydrogen. The reaction solution was taken out of the autoclave and left for approximately 30 minutes. The reaction solution was separated into two layers, the oil phase of the lower layer and the water phase of the upper layer. The methylene chloride solution in the lower layer was isolated and the water phase was extracted with methylene chloride once. The methylene chloride solutions were mixed, dried over anhydrous magnesium sulfate, and concentrated to recover the solvent, whereby 9.8 g of crude (2R)-methylbutanoic acid was obtained. The crude (2R)-methylbutanoic acid was distilled to obtain 9.3 g of purified (2R)-methylbutanoic acid: boiling point 85° C./11 mmHg; GC purity 99.7%; optical purity 94.8% ee; optical rotation [α]_{D}²⁰-19.5 (c 1.04, MeOH); mass spectrum (20 eV, m/e) 29, 41, 55, 56, 57, 73, 74, 87, and 103 (M⁺+1).

### Example 2

### Syntheses of (2R)-methylbutanoic acid Via Process of Recycling Water Phase

10 g (0.1 mol) of tiglic acid (available from Tokyo Kasei Kogyo Co., Ltd.) and 11.3 mg (1×10⁻² mmol) of [RuCl(p-cymene){(R)—(SO₃Na)₂BINAP}]Cl were put in a 200 mL autoclave, and the atmosphere in the autoclave was replaced with nitrogen. 40 mL of degassed distilled diisopropyl ether and 20 mL of degassed distilled water were added to the mixture, and tiglic acid was reacted at 80° C. for 3 hours under the hydrogen pressure of 2.5 MPa. The temperature of the autoclave was lowered to the room temperature, hydrogen was discharged, and nitrogen was flowed in the autoclave for approximately 30 minutes to remove the remaining hydrogen. Then, the reaction solution was ejected from a sampling hole of the autoclave into a 100 mL glass syringe having a needle with the inside diameter of 1.5 mm under nitrogen flow utilizing the nitrogen pressure, and left for approximately 30 minutes. The reaction solution was separated into two layers, the organic phase of the upper layer and the water phase of the lower layer.

The water phase was isolated and returned into the autoclave, and sealed under nitrogen to be reused in the next reaction. On the other hand, the oil phase was isolated, dried over anhydrous magnesium sulfate, and concentrated to recover the solvent, whereby 9.61 g of a residue was obtained. The residue was distilled to obtain 9.3 g of purified (2R)-methylbutanoic acid: boiling point 83° C./10 mmHg; GC purity 99.6%; optical purity 92.5% ee; optical rotation [α]_{D}²⁰-19.2 (c 1.07, MeOH).

Then, a solution of 10 g (0.1 mol) of tiglic acid and 40 mL of degassed distilled diisopropyl ether was added into the autoclave containing the water phase used in the above reaction while blocking the air. The tiglic acid was reacted for 3 hours under the same conditions as the above reaction, and subjected to the aftertreatments in the same manner as above to obtain 10.2 g of crude (2R)-methylbutanoic acid: GC purity 99.47%; enantiomer excess 92.5% ee.

The asymmetric hydrogenation of tiglic acid was repeated 4 times such that the water phase was isolated under nitrogen after the reaction and recycled in the same manner as above.

It took 4 hours and 5 hours to complete the third and fourth reactions recycling the water phase, respectively. It was considered that the reaction rate was lowered because the water phase containing the catalyst was mixed in the organic phase and removed with the organic phase.

The results of the reactions recycling the water phase are shown in Table 1.

TABLE 1

| Number of | | | | Yield of crude | |
|---|---|---|---|---|---|
| recycling | Reaction | Conver- | Selec- | 2-methyl- | Optical |
| water | time | sion | tivity | butanoic acid | purity |
| phase | (h) | (%) | (%) | (g) | (% ee) |
| 0 | 3 | 99.82 | 100 | 9.61 | 92.5 |
| 1 | 3 | 99.47 | 100 | 10.19 | 92.5 |
| 2 | 3 | 98.34 | 100 | 10.67 | 92.3 |
| 3 | 4 | 97.26 | 100 | 10.48 | 92.3 |
| 4 | 5 | 96.7 | 100 | 9.68 | 92.2 |

### Example 3

### Synthesis of (2R)-methylpentanoic acid

11.4 g (0.1 mol) of trans-2-methyl-2-pentenoic acid (available from Tokyo Kasei Kogyo Co., Ltd.) and 59.3 mg (4.5×10⁻² mmol) of [RuI(p-cymene){(R)—(SO₃Na)₂BINAP}]I were put in a 200 mL autoclave, and the atmosphere in the autoclave was replaced with nitrogen. 20 mL of degassed distilled water and 22 mL of degassed methylene chloride were added to the mixture, and trans-2-methyl-2-pentenoic acid was reacted at 80° C. for 6 hours under the same hydrogen pressure as Example 1, to obtain 11.2 g of crude (2R)-methylpentanoic acid. The crude (2R)-methylpentanoic acid was distilled to obtain 10.5 g of purified (2R)-methylpentanoic acid: boiling point 105° C./11 mmHg; GC purity 99.1%; optical purity 89.6% ee; optical rotation [α]_{D}²⁰-17 (c 1.0, MeOH); mass spectrum (20 eV, m/e) 41, 43, 45, 55, 56, 71, 73, 74, 87, 101, and 117 (M⁺+1)

### Example 4

### Synthesis of (2R)-methylhexanoic acid

12.8 g (0.1 mol) of trans-2-methyl-2-hexenoic acid (available from Tokyo Kasei Kogyo Co., Ltd.) and 66 mg (5×10⁻² mmol) of [RuI(p-cymene){(R)—(SO₃Na)₂BINAP}]I were put in a 200 mL autoclave, and the atmosphere in the autoclave was replaced with nitrogen. 89.6 mL of degassed distilled water and 25.6 mL of degassed methylene chloride were added to the mixture, and trans-2-methyl-2-hexenoic acid was reacted at 80° C. for 5 hours under the same hydrogen pressure as Example 1, to obtain 12.9 g of crude (2R)-methylhexanoic acid. The crude (2R)-methylhexanoic acid was distilled to obtain 11.8 g of purified (2R)-methylhexanoic acid: boiling point 116° C./11 mmHg; GC purity 99.4%; optical purity 89.3% ee; optical rotation [α]_{D}²⁰-18.7 (c 1.05, MeOH); mass spectrum (20 eV, m/e) 41, 43, 55, 56, 57, 69, 73, 74, 75, 85, 87, 101, 113, and 131 (M⁺+1).

### Example 5

### Synthesis of (2R)-ethylhexanoic acid

14.2 g (0.1 mol) of 2-ethyl-2-hexenoic acid (available from Aldrich, trans: 94%, cis: 4.83%) and 53 mg (4.66×10⁻² mmol) of [RuI(p-cymene) {(R)—(SO₃Na)₂BINAP}]I were put in a 500 mL autoclave, and the atmosphere in the autoclave was replaced with nitrogen. 210 mL of degassed distilled water and 28.4 mL of degassed methylene chloride were added to the mixture, and 2-ethyl-2-hexenoic acid was reacted at 80° C. for 8 hours under the same hydrogen pressure as Example 1, to obtain 13.9 g of crude (2R)-ethylhexanoic acid. The crude (2R)-ethylhexanoic acid was distilled to obtain 13.5 g of purified (2R)-ethylhexanoic acid: boiling point 125° C./11 mmHg; GC purity 99.1%; optical purity 86.4% ee; optical rotation [α]_{D}²⁰-9.1 (c 1.01, MeOH); mass spectrum (20 eV, m/e) 41, 43, 45, 55, 57, 73, 87, 88, 101, 115, 116, and 145 (M⁺+1).

### Example 6

### Syntheses of (2R)-methylbutanoic acid Via Process of Recycling Water Phase

20 g (0.2 mol) of tiglic acid and 26.3 mg (1×10⁻² mmol) of [RuI(p-cymene){(R)—(SO₃Na)₂BINAP}]I were put in a 200 mL autoclave, and the atmosphere in the autoclave was replaced with nitrogen. 80 mL of degassed distilled water were added to the mixture, and tiglic acid was reacted at 60° C. for 3 hours under the hydrogen pressure of 1.8 MPa. The temperature of the autoclave was lowered to the room temperature, hydrogen was discharged, and nitrogen was flowed in the autoclave for approximately 30 minutes to remove the remaining hydrogen. Then, the reaction solution was ejected from a sampling hole of the autoclave into a 100 mL glass syringe having a needle with the inside diameter of 1.5 mm under nitrogen flow utilizing the nitrogen pressure, and left for approximately 30 minutes. The reaction solution was separated into two layers, the organic phase of the upper layer and the water phase of the lower layer.

The water phase was isolated and returned into the autoclave, and sealed under nitrogen to be reused in the next reaction. On the other hand, the oil phase was isolated, dried over anhydrous magnesium sulfate, and concentrated to recover the solvent, whereby a residue was obtained. The residue was distilled to obtain purified (2R)-methylbutanoic acid. The results are shown in Table 2.

Then, a solution of 20 g (0.2 mol) of tiglic acid was added into the autoclave containing the water phase used in the above reaction and 0.8 mg of [RuI (p-cymene) {(R)—(SO₃Na)₂BINAP}]I while blocking the air. The tiglic acid was reacted for 3 hours under the same conditions as the above reaction, and subjected to the aftertreatments in the same manner as above to obtain (2R)-methylbutanoic acid.

The asymmetric hydrogenation of tiglic acid was repeated 10 times such that the water phase was isolated under ogen after the reaction and recycled in the same manner bove. The results of the reactions recycling the water phase shown in Table 2.

TABLE 2

| Recycle | Time (h) | Conv | % ee | Yield |
|---|---|---|---|---|
| 0 | 3 | 100 | 94.0 | 87.0 |
| 1 | 3 | 100 | 93.8 | 97.6 |
| 2 | 3 | 100 | 93.9 | 98.5 |
| 3 | 3 | 99.1 | 93.9 | 98.1 |
| 4 | 6 | 100 | 93.5 | 98.0 |
| 5 | 6 | 100 | 93.3 | 98.3 |
| 6 | 6 | 100 | 93.4 | 98.7 |
| 7 | 6 | 100 | 93.5 | 98.6 |
| 8 | 12 | 100 | 93.2 | 98.0 |
| 9 | 12 | 99.0 | 93.2 | 98.5 |
| 10 | 24 | 100 | 93.3 | 98.6 |

Recycle 1-10 were carried out by adding catalysts of 3% excess amount of initial quantity to each recycling.

As the number of recycling times increased, the conversion declined, however, the problem was resolved by extending the reaction time. As for the conversion, no advantage of using distilled material was identified, though as for the optical purity, its elevation was seen and maintained 93% ee, even in cases in which the number of recycling times increased.

### Example 7 to 10

The asymmetric hydrogenation was carried out in the same manner as described in Example 1, except that the amount of [RuI(p-cymene){(R)—(SO₃Na)₂BINAP}]I and reaction time were replaced as those in Table 3, and obtained results as described in Table 3.

TABLE 3

| | [Rul(p- | | | | |
|---|---|---|---|---|---|
| | cymene){(R)—(SO3Na)2BINAP}]I | | | | |
| Example | (mg) | Time (h) | Conv | % ee | % Yield |
| 7 | 17.6 | 7 | 100 | 93.1 | 91.7 |
| 8 | 10.5 | 7 | 98.8 | 93.0 | 91.1 |
| 9 | 8.79 | 14 | 98.8 | 92.9 | 91.8 |
| 10 | 5.27 | 24 | 98.6 | 92.1 | 94.9 |

In the methods of the invention, the asymmetric hydrogenation of the α,β-unsaturated carboxylic acid is carried out in water or the two-phase system of water and an organic solvent to obtain a desired optically active carboxylic acid with high optical purity, whereby the methods do not require complicated operations of isolating the produced optically active carboxylic acid and the sulfonated BINAP—Ru complex to be excellent in workability. Further, the methods of the invention can remarkably reduce the costs, can utilize the catalyst efficiently, and are excellent in the workability, because the sulfonated BINAP—Ru complex used in the asymmetric hydrogenation can be recovered and reused without complicated recovering processes. Furthermore, the recovered water phase can be directly reused, and thus, the methods require less labor and costs, thereby further improving the workability.

1. A method for producing an optically active carboxylic acid represented by the formula [2]: 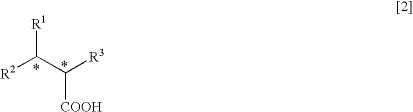
wherein R¹, R² and R³ independently represent a hydrogen atom, an alkyl group, an alkenyl group or an aryl group, the groups may have a substituent, R¹, R² and R³ is not a hydrogen atom simultaneously, R³ is a group other than a hydrogen atom when one of R¹ and R² is a hydrogen atom, R³ is a group other than a hydrogen atom and a methyl group when both of R¹ and R² are hydrogen atoms, and R¹ and R² are different groups other than a hydrogen atom when R³ is a hydrogen atom, and at least one of the two carbon atoms marked with * represents an asymmetric carbon atom, comprising the step of subjecting an α,β-unsaturated carboxylic acid represented by the formula [1]: 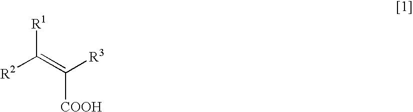
wherein R¹ to R³ have the same meanings as those in the formula [2], in the presence of a sulfonated BINAP—Ru complex represented by the formula [3]:
[RuX(arene) {(SO₃M)₂-BINAP}]X [3]
wherein (SO₃M)₂-BINAP represents a tertiary phosphine represented by the formula [4]: 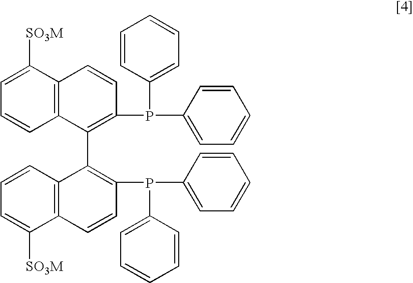
M represents an alkaline metal atom, X represents a chlorine atom, a bromine atom or an iodine atom, and arene represents a benzene or an alkyl-substituted benzene, in an aqueous solvent, to an asymmetric hydrogenation.

2. The method according to claim 1, wherein the aqueous solvent is water or a mixed solvent of water and a water-insoluble organic solvent.

3. The method according to claim 1, wherein the sulfonated BINAP—Ru complex is recovered.

4. The method according to claim 1, wherein the sulfonated BINAP—Ru complex is recycled.

5. A method for producing an optically active carboxylic acid represented by the formula [2]: 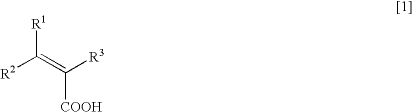
wherein R¹, R² and R³ independently represent a hydrogen atom, an alkyl group, an alkenyl group or an aryl group, the groups may have a substituent, R¹, R² and R³ is not a hydrogen atom simultaneously, R³ is a group other than a hydrogen atom when one of R¹ and R² is a hydrogen atom, R³ is a group other than a hydrogen atom and a methyl group when both of R¹ and R² are hydrogen atoms, and R¹ and R² are different groups other than a hydrogen atom when R³ is a hydrogen atom, and at least one of the two carbon atoms marked with * represents an asymmetric carbon atom, comprising the step of subjecting an α,β-unsaturated carboxylic acid represented by the formula [1]: 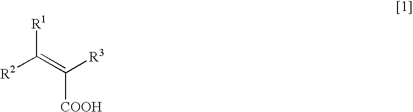
wherein R¹ to R³ have the same meanings as those described above, in the presence of a recovered sulfonated BINAP—Ru complex used in the method according to claim 1 in water or a mixed solvent of water and a water-insoluble organic solvent to an asymmetric hydrogenation.

6. The method according to claim 5, wherein the α,β-unsaturated carboxylic acid is hydrogenated in the presence of an aqueous solution containing the sulfonated BINAP—Ru complex, and the aqueous solution is obtained by separating a water phase from the reaction mixture after the asymmetric hydrogenation in the method according to claim 1.
